(19) United States
(12) Patent Application Publication
(10) Pub. No.: US 2005/0260752 A1
(43) Pub. Date: Nov. 24, 2005

(54) Culture medium containing enhancers of oxidative phosphorylation

(76) Inventors: Martin Graham Wilding, Naples (IT); Brian Dale, Sorrento (IT); Raj Chopra, Westbury, NY (US)

Correspondence Address: MARTIN WILDING;CLINICA VILLA DEL SOLE, VIA MANZONI IS, NAPLES, 80126, IT

(21) Appl. No.: 10/403,409

(22) Filed: Apr. 1, 2003

(51) Int. Cl. C12N 5/00; C12N 5/02

(52) U.S. Cl. 435/373; 435/325

(57) ABSTRACT

The present invention relates to a culture medium suitable for mammalian cell, tissue or embryo growth in vitro in which enhancers of oxidative phosphorylation are included. These enhancers include coenzyme Q, alpha.lipoic acid, acetyl-L-carnitine, alpha.tocopherol, These products are introduced into the medium in a soluble form through the use of either a polysorbate surfactant such as Tween ™ or a preparation containing lecithin and surfactant.

### FEDERAL RESEARCH STATEMENT

[No U.S. federal funds were used in this research]

### BACKGROUND OF INVENTION

The present invention relates to a medium for the in vitro culture of mammalian cells, tissues or embryos. The invention includes a composition of additives such as coenzyme Q (ubiquinone) in a membrane permeable form which augment the levels of oxidative phosphorylation in the tissue, cell line or embryo.

All mammalian cells rely on two metabolic systems to produce energy in the form of adenosine trisphosphate (ATP) which is used for DNA repair and cell division. These two systems are aerobic respiration in the form of oxidative phosphorylation which breaks down pyruvate into carbon dioxide and water and anaerobic respiration which causes the production of lactic acid. The components of the respiratory chain include proteins, coenzymes and other cofactors that are either synthesized in the body or derive from food sources. In vitro cell culture is an artificial system for the production of cell lines, tissues or embryos for experimental, industrial or clinical use. In vitro cell culture is known to be less efficient than in vivo growth. This could be due either to inefficient elimination of waste products from metabolic processes, or from the gradual loss of essential cofactors from the tissue due to reduced levels of metabolism. Essential cofactors include factors essential for mitochondrial metabolism such as coenzyme Q.

Coenzyme Q (ubiquinone) is a group of lipophilic quinones which have the ability of transferring reducing equivalents or electrons within a lipid phase of cellular membranes. Coenzyme Q therefore helps to maintain the energy creating (ATP-producing) activity of mitochondria by its activity as an electron carrier within the electron transport chain of cells, tissues or embryos. Coenzyme Q also acts as an antioxidant. Reduced coenzyme Q appears to function as part of a complex chain of antioxidant activity. Another important role for coenzyme Q is in the reduction of radicals of .alpha.-tocopherol and ascorbate formed when these antioxidants are oxidised by oxygen of carboxyl radicals. There are no natural enzymes for the reduction of tocopherol radical or external ascorbate radical, but naturally occurring enzymes that reduce coenzyme Q are present in all membranes, therefore enabling the recycling of tocopherol and ascorbate.

Alpha.lipoic acid is a vitamin-like substance which is a coenzyme for the pyruvate dehydrogenase complex in the mitochondrial matrix. It is an essential cofactor for metabolism in .alpha.ketoacid dehydrogenase reactions. .alpha.lipoic acid usually exists as lipoamide covalently attached to lysine residues of the enzyme complexes. It functions in the transfer of two-carbon fragment from .alpha.hydroxyethylthiamin pyrophosphate to acetyl CoA, and is reduced in the process. The reduced form of .alpha.lipoic acid is dihydrolipoic acid (DHLA), which may have an antioxidant effect. It may prevent lipid peroxidaion by reducing glutathione which in turn recycles vitamin E. DHLA has also been demonstrated to be a free oxygen radical scavenger to reduce peroxyl, ascorbyl and chromanoxyl radicals, and to inhibit singlet oxygen.

Acetyl-L-carnitine is the acetyl ester of carnitine, a biological compound which plays a role in the transport of fatty acids from the cytosol into the mitochondrial matrix of B-oxidation. Acetyl-L-carnitine modulates the metabolism of sugars, lipids and amino acids through the regulation of the intracellular concentration of acetyl-CoA, thus playing a key role in cellular energy production.

Glutathione (L-gamma-glutamyl-L-cysteineyl-glycine; GSH) is an endogenous thiol that detoxifies reactive oxygen species. It is critical to cell viability and the glutathione redox cycle is a primary antioxidant defense system within the mitochondrial matrix.

The object of the invention is to provide a culture medium for mammalian cells, tissues or embryos including factors required for mitochondrial metabolism such as coenzyme Q, .alpha.lipoic acid and glutathione, these introduced into the medium in a water soluble, bioavailable form.

### SUMMARY OF INVENTION

The present invention relates to a culture medium for mammalian cells, tissues or embryos including factors required for mitochondrial metabolism such as coenzyme Q, .alpha.lipoic acid and glutathione, these introduced into the medium in a water soluble, bioavailable form through the use of a polysorbate surfactant such as Tween ™ or Span ™. The medium referred to in the present invention should contain a final concentration of coenzyme Q in the region of 0.0001% to 0.005% by weight of the final composition. Other components of the medium should be present in the medium in a suitable amount as determined by the use to be made of the medium.

Coenzyme Q, being a highly insoluble compound in aqueous solutions, is introduced into the medium in a bioavailable form in a composition containing coenzyme Q in the range of 2% to 15% by weight in either a mixture of primary surfactant such as Tween™ preferable polysorbate 80 and optionally a secondary surfactant such as Span™, together with a mixture of phospholipids (eg hydroxylated lecithin) or a composition containing a primary and if necessary a secondary surfactant together with a mixture of medium chain triglycerides, phospholipids and glyceryl ester. This soluble coenzyme Q preparation is then diluted into the culture medium of interest in the range of between 1 part in 5000 to 1 part in 10000 to give the required concentration of coenzyme Q.

### BRIEF DESCRIPTION OF DRAWINGS

FIG. 1 Structure of Q₁₀

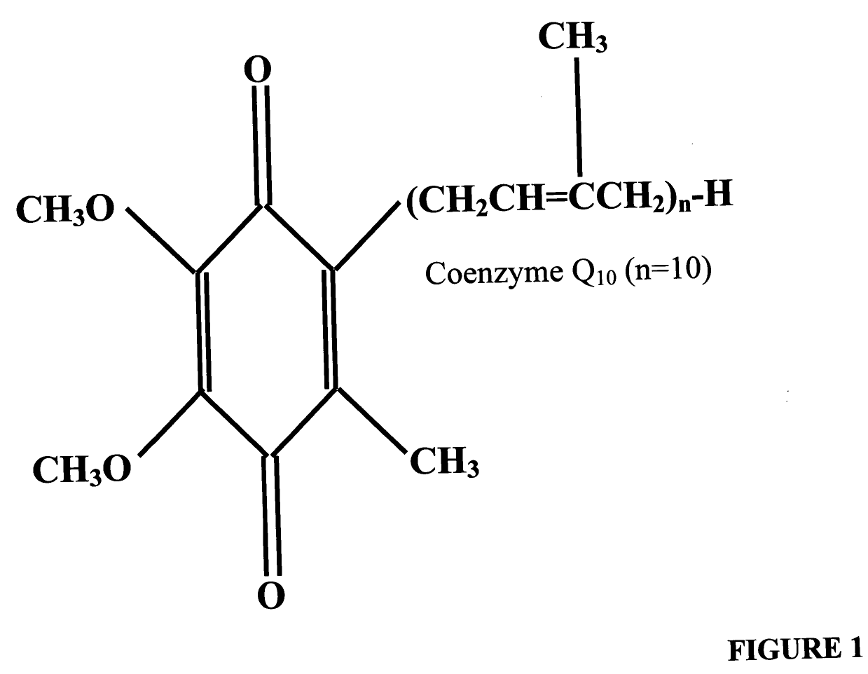

FIG. 2 Structure of Tween 80 ™

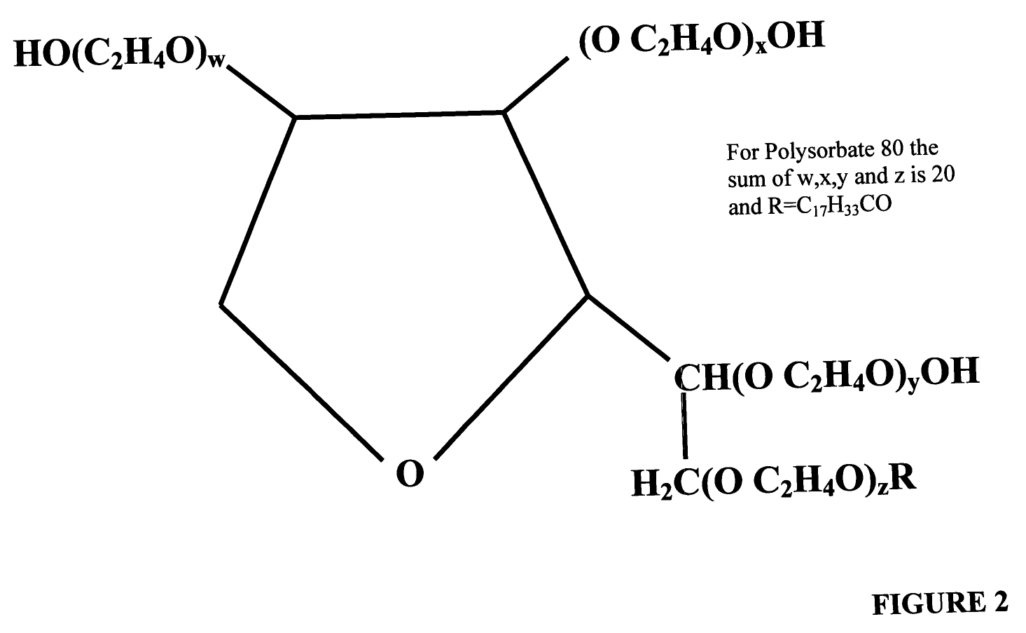

FIG. 3 Structure of Span 20 ™

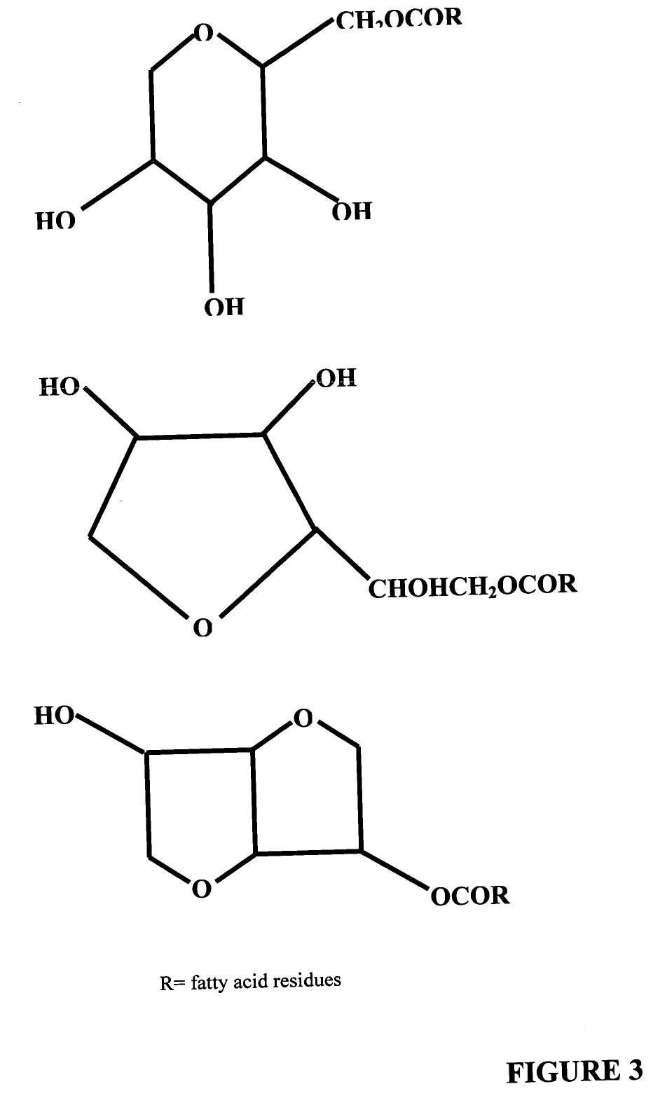

### DETAILED DESCRIPTION

Coenzyme Q or the ubiquinones are terms used to describe the group of lipid soluble benzoquinones involved in mitochondrial electron transport. The compounds are be described as: coenzyme Q.sub.N, where N is 1-12 or ubiquinone (x) where x designates the total number of carbon atoms in the side chain and can be any multiple of 5. The preferred ubiquinone for use in the present invention is coenzyme Q.sub.10:

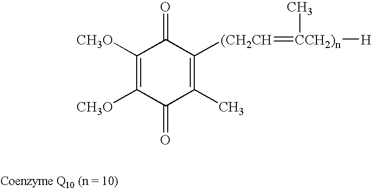

The amount of ubiquinone which is included in the compositions described herein ranges from 0.0001% to 0.005% by weight of the final product.

Surfactants or emulsifiers refer to the compounds used to promote the solubility of the ubiquinone. These are used in combination with phospholipids which further promote the solubility of the ubiquinone, and other triglycerides or vegetable oils may also be employed. The primary surfactants used in the present invention will primarily include polysorbate surfactants (Tween™):

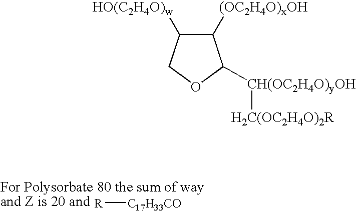

The Tween™ surfactants are oleate esters of sorbitol and anhydrides of sorbitol copolymerised with a number of moles of ethylene oxide per mole of sorbitol or sorbitol anhydride. These surfactants are highly dispersable in water. The preferred Tween™ surfactant herein is a sorbitan mono-9-octadeconate poly(oxy-1,2-etheandiyl) derivative commonly known as Tween™ 80 or polysorbate 80. The final amount of surfactant in the present invention ranges from 0.001% to 0.01%. In certain conditions minor amounts of secondary surfactants such as the Span ™ surfactants may also be added to the composition. Span™ surfactants are partial esters of common fatty acids such as lauric acid, palmitic acid, stearic acid and oleic acids and hexitol anhydrides such as hexitans and hexides derived from sorbitol. The basic structure of such compounds is described below:

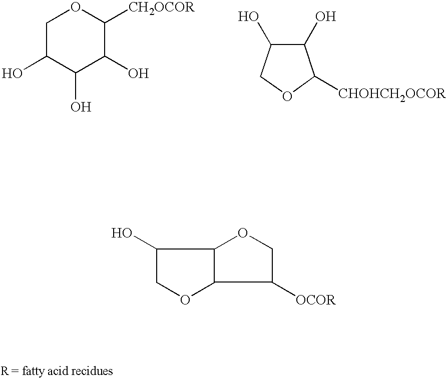

The Span™ surfactants are generally oil soluble, however such surfactants work in tandem with the more water-soluble Tween™ surfactants to solubilise the ubiquinols.

Phospholipids refer to compounds of a lipid-like, amphipathic nature which preferably has a hydrophobic chain together with a hydrophilic, anionic residue. Phospholipids are used to further enhance the solubility of the preparation, and are especially useful where large amounts of ubiquinone are required. Preferred phospholipids include phosphatidylcholine, phosphatidylethanolamine, distearolyphosphatidylcholine, phosphatidylserine, phosphatidylglycerol, phosphatidic acid, phosphatidylinositol, sphingomyelin and lecithin. Where included, the amount of phospholipids generally falls within the range of 0.1% to 25% by weight.

The term “cell”, “embryo” or “tissue” used in the present specification describes any material for which in vitro culture is applied to enable the multiplication or differentiation outside the body. Such materials are oocytes or embryos, stem cells, somatic cells, tissue of any organ or derived from any organ.

The term “effective amount” used throughout the specification describes the amount of compound or components that are added to the composition to produce an intended effect or favourable result, whether that result relates to the physiological effect of the compound or its ability to function for an intended alternative use for example a surfactant or bioactive agent such as tocopherol (including vitamin E or vitamin E ester) which promotes the solubility or bioavailability of the ubiquinone or other bioactive agent.

The term “bioactive agent” is used throughout the specification to indicate a compound in addition to ubiquinone (i.e. other than a coenzyme Q analog) which is added to the composition in an effective amount. Bioactive agents for use within the present invention may include reduced glutathione, L-cysteine, N-acetyl cysteine, ascorbate and vitamin C esters, vitamin A (retinol, retinoic acid) and vitamin A esters, carotenoids, flavinoids, L-carnitine, acetyl-L-carnitine, propionyl L-carnitine, riboflavin, niacinamide, NADH, NADPH. Preferred bioactive reagents for the purposes of the present invention consist of L-carnitine, acetyl-L-carnitine, vitamin E or vitamin E esters, .alpha.lipoic acid and glutathione. These secondary bioactive agents may be present in the composition in the range 0.1% to 20% by weight.

| Component | M.W. | Final conc | | Mg/l |
|---|---|---|---|---|
| NaCl | 53.44 | 116 | mM | 6800 |
| KCl | 74.55 | 5.4 | mM | 400 |
| MgSO₄.7H₂O | 120.4 | 2 | mM | 200 |
| CaCl₂ | 111.0 | 2 | mM | 264.9 |
| NaHCO₃ | 84.01 | 25 | mM | 2200 |
| NaH₂PO₄.2H₂O | 268.1 | 1.3 | mM | 158.3 |
| D-Glucose | 180.2 | 5.5 | mM | 1000 |
| sodium lactate | 112.1 | 1 | mM | 112.1 |
| sodium pyruvate | 110 | 10 | mM | 1100 |

The term “culture medium” is used throughout the present specification to refer to the aqueous medium consisting of salts and carbohydrates prepared to a defined osmolarity and pH which is used for the in vitro culture of mammalian cells, tissues or embryos. Such media can be prepared both in the laboratory by any practitioner skilled in the art and are also commercially available. Various formulations of culture medium are available and examples may include but not be limited to Earles salts for which the basic formula is herein presented: To prepare the basic culture medium according to the present invention, the components of the medium are added in the required composition to purified water at room temperature, and the pH of the final solution balanced to 7.3-7.5 with NaOH. The Osmolarity is then checked, and brought to 280 mOsm-300 mOsm with the addition of water or NaCl. After sterilisation, the medium is designed to be used in an atmosphere of 35-40° C. and 4-6% CO₂.

The basic ubiquinone-containing medium is prepared as follows:

### EXAMPLE 1

### Liquid form of Coenzyme Q₁₀

| Wt. range | Components | W/w |
|---|---|---|
| 0.05%-15% | Coenzyme Q₁₀ | 7% |
| 0.1%-50% | Tween ™ 80 | 38% |
| 0.1%-50% | Span ™ 20 | 5% |
| 0.5%-35% | Medium chain triglycerides | 33% |
| 0.01%-25% | Vitamin E alcohol or acetate | 17% |

Procedure:1)Add Span™ 20 to the medium Chain Triglycerides in a jacketed mixing vessel. Heat to 130±5° F. with constant stirring at 160 RPM for approx. 2 hours or until dissolved.

2)Add Tween™ 80 to the above solution with constant stirring while maintaining the temperature at 130±5° F. and mix for at least 60 minutes.

3)Screen the Co Q₁₀ powder through a 100-mesh screen into the liquid blend while stirring and maintaining the temperature at 130±5° F. Keep stirring until a clear solution is obtained (60-90 minutes). Then add the vitamin E alcohol or acetate and stir for an additional 30 minutes.

4)Remove the source of heat. Cool with continuous mixing.

5)Sterilise by filtration or other suitable technique. Store in an air and light-resistant container.

6)Test for assay of CoQ₁₀, dissolution of CoQ₁₀ etc.

### EXAMPLE 2

### Liquid form of Coenzyme Q₁₀

| Wt. range | Components | W/w |
|---|---|---|
| 0.05%-15% | Coenzyme Q₁₀ | 7% |
| 0.1%-50% | Tween ™ 80 | 29% |
| 0.1%-50% | Span ™ 20 | 5% |
| 0.5%-35% | Medium chain triglycerides | 32.5% |
| 0.01%-25% | Vitamin E alcohol or acetate | 17% |
| 0-25% | Hydroxylated lecithin (or high | 9.5% |
| | PC lecithin | |

Procedure:1)Add Span™ 20 to the medium Chain Triglycerides in a jacketed mixing vessel. Heat to 130±5° F. with constant stirring at 160 RPM for approx. 2 hours or until dissolved.

2)Add Tween™ 80 to the above solution with constant stirring while maintaining the temperature at 130±5° F. and mix for at least 60 minutes. Add the hydroxylated lecithin and continue to stir for at least 90 minutes.

3)Screen the Co Q₁₀ powder through a 100-mesh screen into the liquid blend while stirring and maintaining the temperature at 130±5° F. Keep stirring until a clear solution is obtained (60-90 minutes). Then add the vitamin E alcohol or acetate and stir for an additional 30 minutes.

4)Remove the source of heat. Cool with continuous mixing.

5)Sterilise by filtration or other suitable technique. Store in an air and light-resistant container.

6)Test for assay of CoQ₁₀, dissolution of CoQ₁₀ etc.

Formulations of Coenzyme Q₁₀ prepared according to examples such as the two described above are then diluted into culture medium in a range of 1 part to 5000-10000 parts culture medium. It must be stated that those skilled in the art may use varying techniques or methods of preparing the above formulations, but the above examples and descriptions are in no way limiting. Variations in the details presented herein may be made without departing from the spirit and scope of the present invention which is defined under the following claims:

1. A culture medium for use with mammalian cells, tissues or embryos consisting essentially of vital salts in physiological concentrations, carbohydrate and amino acid energy sources and further additives designed to enhance oxidative phosphorylation and reduce the generation of reactive oxygen species, these consisting of coenzyme q and secondary bioreactive reagents.

2. The composition according to claim 1, wherein an effective amount of these additives are solubilised in a composition containing:
i. An effective amount of ubiquinone
ii. An effective amount of primary surfactant within the range of about 0.1% to about 50% by weight
iii. An effective amount of a secondary surfactant within the range of about 0.1% to 50% by weight
iv. A triglyceride in an amount ranging from about 0.1% to about 25% by weight
v. An effective amount of a secondary bioactive reagent or reagents in an amount ranging from about 0% to 25% by weight.
This composition being added to the culture medium in an amount ranging from about 1 part in 5000 to 1 part in 10000.

3. The composition according to claim 2 wherein said primary surfactant consists of a polysorbate surfactant such as polysorbate 80 (Tween ™) in an amount ranging from about 0.1% to about 50% by weight.

4. The composition according to claim 2 wherein said secondary surfactant consists of Span™ surfactant in an amount ranging from about about 0.1% to 50% by weight.

5. The composition according to claim 2 wherein said triglyceride is a mixture of medium chain triglycerides comprising about 0.1% to about 25% by weight.

6. The composition according to claim 2 wherein said secondary bioreactive reagent or reagents are selected from the group of .alpha.tocopherol, tocopherol esters, .alpha.lipoic acid, ascorbate esters, retinal, retinoic acid, retinal acetate, retinal, L-carnitine, acetyl-L-carnitine, glutathione or mixtures thereof.

7. The composition according to claim 1, wherein an effective amount of these additives are solubilised in a composition containing:
i. An effective amount of ubiquinone
ii. An effective amount of primary surfactant within the range of about 0.1% to about 50% by weight
iii. An effective amount of a secondary surfactant within the range of about 0.1% to 50% by weight
iv. A triglyceride in an amount ranging from about 0.1% to about 25% by weight
v. An effective amount of phospholipid ranging from about 0.1% to 25% by weight.
vi. An effective amount of a secondary bioactive reagent or reagents in an amount ranging from about 0% to 25% by weight.
This composition being added to the culture medium in an amount ranging from about 1 part in 5000 to 1 part in 10000.

8. The composition according to claim 7 wherein said primary surfactant consists of a polysorbate surfactant such as polysorbate 80 (Tween ™) in an amount ranging from about 0.1% to about 50% by weight.

9. The composition according to claim 7 wherein said secondary surfactant consists of Span™ surfactant in an amount ranging from about about 0.1% to 50% by weight.

10. The composition according to claim 7 wherein said triglyceride is a mixture of medium chain triglycerides comprising about 0.1% to about 25% by weight.

11. The composition according to claim 7 wherein said phospholipid is selected from the group containing phosphatidylcholine, phosphatidylethanolamine, distearolyphosphatidylcholine, phosphatidylserine, phosphatidylglycerol, phosphatidic acid, phosphatidylinositol, sphingomyelin and hydroxylated lecithin within the range of 0.1% to 25% by weight.

12. The composition according to claim 7 wherein said secondary bioreactive reagent or reagents are selected from the group of .alpha.tocopherol, tocopherol esters, .alpha.lipoic acid, ascorbate esters, retinal, retinoic acid, retinal acetate, retinal, L-carnitine, acetyl-L-carnitine, glutathione or mixtures thereof.

13. An improved liquid form of Coenzyme Q₁₀ solution of the type containing an aqueous solution of Coenzyme Q₁₀ in the range of 0.05% to 15.0% by weight, Tween™ 80 (Polysorbate 80) in the range of 0.1% to 50%, Medium Chain Triglycerides in the range of 0.5% to 35% and Vitamin E alcohol (or acetate) in the range of 0.01% to 25%, wherein the improvement comprises:
replacing tributyrin (Glyceryl tributyrate) in the range of 0.1% to 50% with Span™ 20 in the range of 0.1% to 50%.

14. An improved liquid form of Coenzyme Q₁₀ solution of the type containing an aqueous solution of Coenzyme Q₁₀ in the range of 0.1% to 15.0% by weight, Tween™ 80 (Polysorbate 80) in the range of 0.1% to 50%, Medium chain triglycerides in the range of 0.5% to 35%, Hydroxylated lecithin (or high PC lecithin) in the range of 0.0% to 25% and Vitamin E alcohol (or acetate) in the range of 0.01% to 25%, wherein the improvement comprises:
replacing Tributyrin (Glyceryl tributyrate) in the range of 0.1% to 50% with Span™ 20 in the range of 0.1% to 50%.
